(19) United States
(12) Patent Application Publication
(10) Pub. No.: US 2014/0360503 A1
(43) Pub. Date: Dec. 11, 2014

(54) PATIENT INTERFACE DEVICE

(71) Applicant: KONINKLIJKE PHILIPS N.V., EINDHOVEN (NL)

(72) Inventors: Steven Ernest Franklin, Eindhoven (NL); Sander Theodoor Pastoor, Utrecht (NL); Franciscus Joannes Leonardus Everaerts, Weert (NL)

(73) Assignee: KONINKLIJKE PHILIPS N.V., EINDHOVEN (NL)

(21) Appl. No.: 14/362,896

(22) PCT Filed: Nov. 13, 2012
(86) PCT No.: PCT/IB2012/056377

§ 371 Date: Jun. 4, 2014

Related U.S. Application Data
(60) Provisional application No. 61/569,310, filed on Dec. 12, 2011.

(51) Int. Cl. A61M 16/06; A61M 16/00

(52) U.S. Cl. CPC A61M 16/0683; A61M 16/0057; A61M 16/0605; A61M 2205/04; A61M 2210/02
USPC 128/205.25

(57) ABSTRACT

The present invention relates to a patient interface device and system for delivering continuous airway pressure to a patient (14). The patient interface system comprises a patient interface device with a patient interface (12) and at least one connector pair (22), comprising itself two connector parts, attached to the patient interface (12) and the patient (14) via a tissue anchor, respectively. One of the connector parts comprises a magnet and the other one a magnetically responsive element. Both connector parts are attracted to one another through the magnetic forces.

### FIELD OF THE INVENTION

The present invention relates to a patient interface device and a patient interface system for delivering a gas to a patient. Such patient interfaces are often referred to as facial masks or respiratory interface devices. The present invention relates further to a method for attaching a patient interface device to a patient.

### BACKGROUND OF THE INVENTION

For several chronic disorders and diseases, e.g. Obstructive Sleep Apnea (OSA), a long-term attachment of a patient interface to a patient is necessary or at least advisable. Therein, airway pressure is often used. For example, sleep apnea can be treated in an extremely effective way by using airway pressure (e.g. CPAP, BiPAP). Therefore, the afore-mentioned long-term use of the patient interface takes place during the sleeping time of the patient.

As an example for this application, four basic types of patient interfaces shall be mentioned:
nasal masks, which fit over the nose and deliver a gas through the nasal passages,
oral masks, which fit over the mouth and deliver a gas through the mouth,
full face masks, which fit over both, the nose and the mouth, and deliver a gas to both, and
nasal pillows, which are regarded as masks as well within the scope of this invention and which consist of small nasal inserts that deliver the gas directly into the nasal passages.

If the term “mask” is used herein, this shall not be understood as a limitation to this special embodiment rather than referring to patient interfaces as described herein in general, where applicable and if not stated otherwise.

An example for a patient interface designed as a full face mask for the usage with continuous airway pressure is described in U.S. Pat. No. 7,640,933 B1.

Resulting from the afore-mentioned long-term usage of those patient interfaces, an interface attachment can often be the reason for discomfort of the patient. This is especially disturbing for the patient if the patient interface is used, for example, for the mentioned treatment of sleep apnea. Especially at that time, i.e. in sleep, a patient wants to and should have no disturbing influences.

Commonly known masks all show a fixation to the patient by using some sort of headgear. Although the mask and headgear can vary in size, shape and material, the headgear normally covers significant parts of the whole head and is designed of relatively robust materials due to the use in a continuous airway pressure application.

### SUMMARY OF THE INVENTION

It is an object of the present invention to provide a patient interface device, which is attachable in such a way to a patient that it avoids the use of cumbersome straps and headgear, reducing the discomfort and inconvenience a patient feels by wearing the attached patient interface in a long-term application.

According to an aspect of the present invention, a patient interface device is provided, comprising:
a patient interface for delivering a gas to a patient, and
at least one first connector part of at least one connector pair, said at least one connector pair comprising two connector parts and being provided for arranging said patient interface to the patient,
wherein said at least one first connector part comprises at least one magnet or at least one magnetically responsive element.

According to another aspect of the present invention, a tissue anchor is provided, being fixable to a patient, comprising:
at least one second connector part of at least one connector pair, said at least one connector pair comprising two connector parts and being provided for arranging a patient interface to the patient,
wherein said at least one second connector part comprises at least one magnet or at least one magnetically responsive element.

Within this invention, the second connector part may be formed by the tissue anchor itself, may be a part of the tissue anchor or may be arranged at the tissue anchor. Although the tissue anchor may comprise several second connector parts corresponding to one first connector part, embodiments wherein the tissue anchor comprises one second connector part that corresponds to one first connector part is preferred. This does include embodiments, wherein the one second connector part is formed by several elements. Preferably, the first connector part of the patient interface device and the second connector part comprised by the tissue anchor are forming the connector pair.

The magnet used in the tissue anchor can be a permanent or an electro-magnet, although embodiments comprising a permanent magnet are preferred.

According to another aspect of the present invention, a patient interface system is provided, comprising:
a patient interface device of the kind mentioned before and
at least one tissue anchor of the kind mentioned before,
wherein the number of tissue anchors comprising the at least one second connector part corresponds to the number of the at least one first connector parts of the patient interface device, thereby forming a respective number of connector pairs.

Contrary to the proposed patient interface system the proposed patient interface device does not comprise elements that are fixable or fixed to the patient.

According to another aspect of the present invention, a method for attaching a patient interface device of the kind mentioned before to a patient is provided, comprising:
arranging the patient interface at the patient and
magnetically connecting at least one first connector part of at least one connector pair to at least one second connector part of said at least one connector pair,
wherein said at least one first connector part is comprised by said patient interface device and said at least one second connector part is comprised by a tissue anchor, said tissue anchor being fixed to the patient.

Preferred embodiments of the invention are defined in the dependent claims. It shall be understood that the patient interface system may have the same or similar preferred embodiments as explained hereinafter and as defined in the dependent claims for the patient interface device and the tissue anchor. It shall be further understood that the claimed method has similar and/or identical preferred embodiments as the claimed device and as defined in the dependent claims.

The word “magnet” mentioned here is to be understood within the scope of the present invention as either permanent magnet or electro-magnet, if not stated otherwise.

Attaching the patient interface to the patient using a tissue anchor located and fixed at the patient avoids the necessity of a huge and inconvenient headgear normally used in therapies like continuous airway pressure therapy. Since magnetic fields have the ability to pass and act through skin tissue hiding the tissue anchor portion under the skin of the patient is possible. Thus, such a tissue anchor fixed to the patient has a higher comfort than other ways of anchor fixation in which the anchor can be seen. These are in contrast to the present invention necessary if the anchor has to be in direct contact with the patient interface or a coupling element of the patient interface device.

Within this invention it is preferred to provide magnets on both, the patient, i.e. the tissue anchor, and the patient interface device, or to provide a magnet on just one of both and arrange a magnetic responsive element, which is not a magnet, on the other side. Having one passive, i.e. non-magnet side, leaves this side inactive and reduces the interaction with other metal things of the every-day life if the patient interface is not worn. Having magnets on both sides may result in a stronger hold and may further aid by directing the attachment of the patient interface to the patient. This is due to the effect that magnets directed to each other with the same pole repel themselves, and may be realized by positioning the magnets used for the first and second connector part accordingly.

The term “magnetic responsive element” encompasses in general elements being a magnet themselves or, preferably, non-magnets that show a response to a magnetic field, like ferromagnetic or ferrimagnetic materials. Such a response is preferably an attraction to a magnet.

Wherein the usage of electro-magnets as magnets allows to turn the magnetic field used to arrange the patient interface at the patient on and off, application of a permanent magnet makes it possible to use the patient interface device of the present invention without the need of a power source.

According to a preferred embodiment of the device of the present invention, the first connector part comprises a plurality of magnets or a plurality of magnetic responsive elements. Using two or more magnets or magnetic responsive elements for the first connector part provides a better attachment of the patient interface to the patient. This is due to the possibility of a better adaptation of the first connector part to the mostly round shape of the patient interface and the patient's face.

According to another embodiment of the device of the present invention, the at least one magnetically responsive element of the at least one first connector part comprises a ferromagnetic or ferrimagnetic material. Usage of a ferromagnetic or ferrimagnetic material as part of the magnetically responsive element provides a passive part of the patient interface device that does in general not interact with other (metal) things when the patient interface device is not used.

According to another embodiment of the device of the present invention, the at least one magnet is at least one permanent magnet or at least one electro-magnet. Permanent magnets provide magnetic fields independent of a power source. Electro-magnets allow for activation and deactivation of the magnetic field and thus for easy attachment and detachment of the patient interface to the patient. Further, if the patient does not wear the patient interface the magnetic field may be disabled in order not to interact with other things.

According to another embodiment of the device of the present invention, the at least one first connector part is arranged at the patient interface. This way an attachment of the patient interface to the patient can easily be realized while simultaneously providing a compact patient interface design.

According to another embodiment of the device of the present invention, the patient interface device comprises a plurality of first connector parts of a plurality of connector pairs. Having several first connector parts, the patient interface may be attached more evenly to the patient's face. Further, the single force necessary to connect one first with a corresponding second connector part of a connector pair may be kept to a minimum. Thereby, irritations to the patient, especially to the patient's skin, may be avoided while wearing the patient interface. These might in contrast occur, if the ratio of the number of the first connector parts and accordingly the number of the connector pairs to the weight of the patient interface is such that a higher single force is needed to keep the patient interface at the patient. The result would be a higher pressure on the patient's skin. For this embodiment, the patient interface device comprises preferably 2 to 10, more preferably 2 to 5 first connector parts.

According to an embodiment of the tissue anchor of the present invention, the at least one second connector part comprises a plurality of magnets or a plurality of magnetically responsive elements. Using two or more magnets or magnetic responsive elements for the second connector part provides a better attachment basis for the patient interface. This is due to the possibility of a better adaptation of the second connector part to the round shape of the patient's face.

According to another embodiment of the tissue anchor of the present invention, the at least one magnetically responsive element of the at least one second connector part comprises a ferromagnetic or ferromagnetic material. Usage of a ferromagnetic or ferrimagnetic material as part of the magnetically responsive element provides a passive part of the tissue anchor that does in general not interact with other (metal) things when the patient interface is not worn.

According to another embodiment of the tissue anchor of the present invention, the anchor is adapted to be arranged subcutaneously, more preferably at bone tissue. The subcutaneous arrangement hides the tissue anchor so that it is not visible if the patient does not wear the patient interface. Arranging the anchor at the bone tissue provides a strong fixation to the patient. In such an arrangement, the tissue anchor is preferably a bone anchor.

According to another embodiment of the tissue anchor of the present invention, the anchor further comprises at least one docking station being adapted to be attached to bone tissue and wherein the at least one second connector part is releasable accommodated by the docking station, wherein the docking station is preferably composed of non conductive materials. The usage of a docking station allows for easy installation and retrieval of the second connector part at and from the patient. Considering the magnetic or at least conductive material used for the second connector part, a removal of these is necessary if the patient has to be examined in an MRI for example. For the docking station itself being non-conductive, this is easily achieved. Preferably, the tissue anchor comprises one docking station for one second connector part.

Part of the invention is also that any of the elements of the connector pair may be fixated in place using e.g. an adhesive. For instance, the tissue anchor may be comprised in an adhesive plaster to be attached to a person's skin. The tissue anchor fixated in this way could then interact with a first connector part comprised in a patient interface to fixate the patient interface on the person's face. It is also possible that a plurality of tissue anchors is fixated on a patient to allow adjustment of the position of a patient interface relative to the patient's face by a having first connector part comprised in a patient interface interact with an appropriate subset of the plurality of tissue anchors. The plurality of tissue anchors may be comprised in one or more adhesive plasters to be attached to a patient's skin.

In all mentioned embodiments of the present invention, the patient interface device is preferably designed to deliver pressurized gas to a patient. Generally, however, the proposed mask apparatus is adapted to deliver any kind of gas to a patient. Hence, whenever “air” or “a gas” is mentioned herein, this term shall not be understood as limited to pure “air”, but shall include any kind of air or gas that shall be delivered to a patient, e.g. air enriched in oxygen, a gas mixture comprising oxygen and a medicine.

### BRIEF DESCRIPTION OF THE DRAWINGS

These and other aspects of the invention will be apparent from and elucidated with reference to the embodiment described hereinafter. In the following drawings

FIG. 1 shows a schematic perspective view of a patient wearing a patient interface device according to the present invention,

FIG. 2 shows a schematic side view of the patient of FIG. 1,

FIG. 3 shows a schematic perspective view of a patient interface,

FIG. 4 shows a schematic partly sectional view of an area around the nose of the patient,

FIG. 5 shows a schematic view of a tissue anchor comprising a second connector part and

FIG. 6 shows a schematic view of the sole anchor.

### DETAILED DESCRIPTION OF THE INVENTION

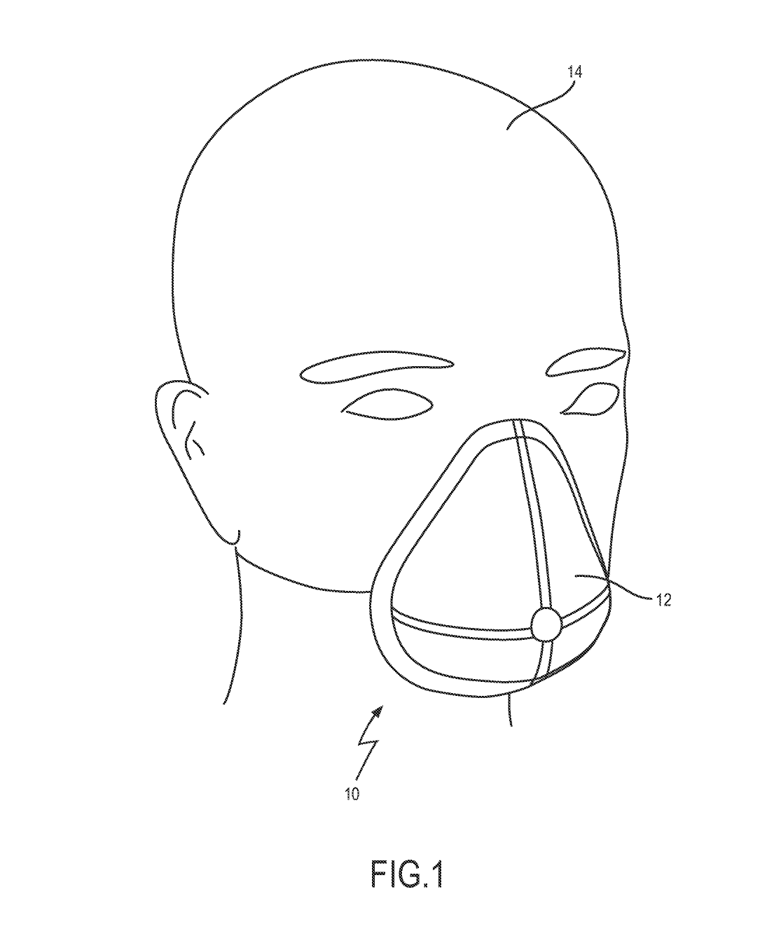

An embodiment of a patient interface device according to the present invention is shown throughout and described with the help of FIGS. 1 to 6 and is designated in its entirety by the reference numeral 10.

The patient interface device 10 comprises a patient interface 12. This patient interface 12 may be worn by a patient 14 as can be seen in FIG. 1. As an exemplary type of application, this patient interface is used for the treatment of sleep apnea by applying continuous positive airway pressure (CPAP) to the patient 14. For this, as well as for other possible applications, the patient interface 12 has to be attached sufficiently to the patient 14.

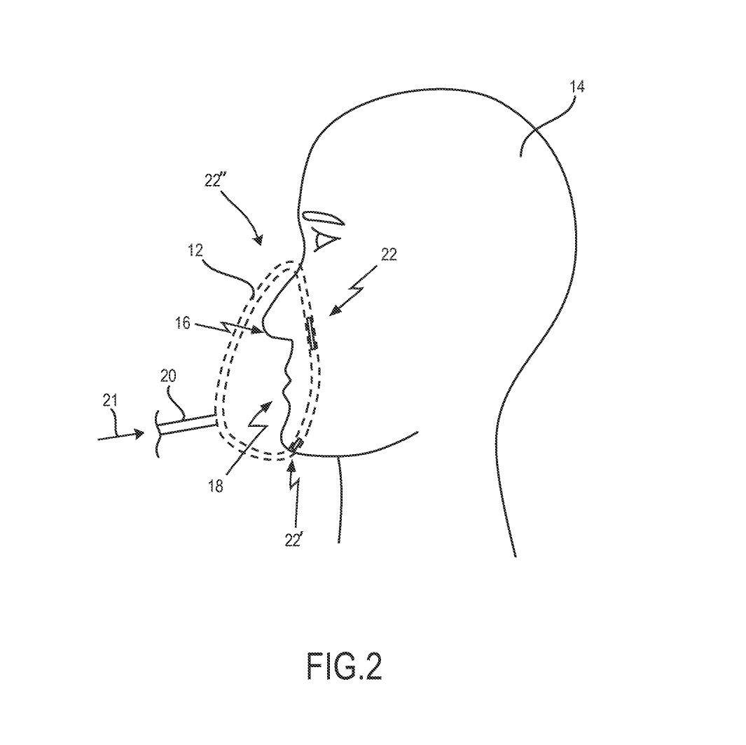

The patient interface 12 covers in this particular embodiment the nose 16 and mouth 18 of the patient 14, which can be seen in FIG. 2. This type is therefore called a full face mask. In other types of patient interfaces, like the aforementioned nasal masks, oral masks and nasal pillows, only one of both is covered. However, the kind of patient interface has no influence on the invention as it can be applied to all kinds of patient interfaces used as respiratory interfaces.

For clarity reasons, the patient interface 12 is shown in FIG. 2 just by dashed lines so that the positioning on the face of the patient 14 can be seen.

The patient interface device 10 further comprises a hose 20. The hose 20 is attached to the patient interface 12 and is used to supply the gas to the patient interface 12 and therefore to the patient 14. This is indicated by an arrow 21. Although, air is an option for the gas supplied to the patient 14 in this case, any other gas that shall be supplied to a patient may be used with the patient interface device 10 within the present invention.

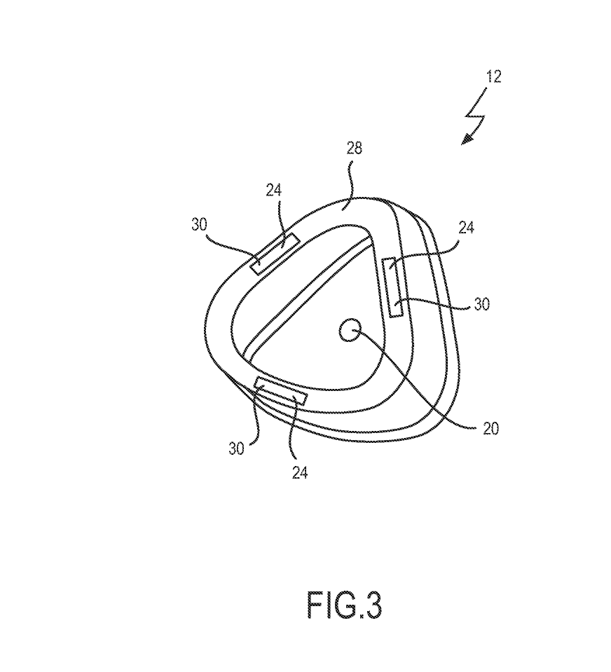

For attaching the patient interface 12 to the patient 14 the patient interface device 10 comprises in this embodiment three first connector parts 24. This is shown in FIG. 3. These connector parts 24 are part of three connector pairs 22, 22′ and 22″. Connector pairs 22 and 22′ can be seen in FIG. 2, whereas connector pair 22″ is just indicated by an arrow as it is arranged on the back side of the face of the patient 14 with respect to the representation of FIG. 2.

Each connector pair 22, 22′, 22″ comprises the first connector part 24 and a second connector part 26. In this embodiment, the first connector part 24 is arranged on the patient interface 12. Referring to FIG. 3, the patient interface 12 comprises a rim 28. This rim 28 is the portion of the patient interface 12 that gets in contact with the patient 14 once the patient interface 12 is attached to the patient 12. On this rim 28 three first connector parts 24 are arranged. These first connector parts 24 are designed as magnets 30. In this particular embodiment, these magnets 30 are permanent magnets. However, an embodiment wherein those magnets 30 are designed as electro-magnets lies also within the scope of the present invention and works basically in the same way. Also, each first connector part 24 comprises one magnet 30. An embodiment, wherein each connector part 24 comprises a plurality of magnets 30 lies also within the scope of the present invention. A plurality of magnets 30 might be helpful if the first connector part 24 has to be accommodated to a certain design of the patient interface 12.

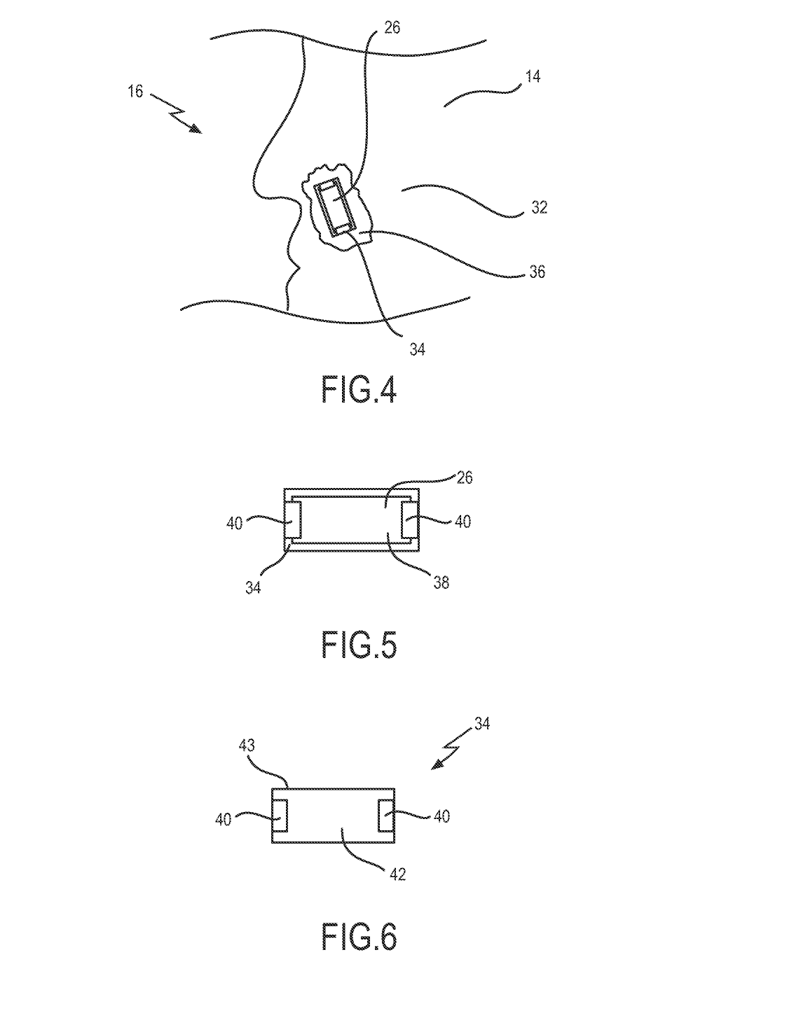

The second connector parts 26 are arranged on the patient 14. In this embodiment, three connector parts 26 are fixedly arranged to the patient 14. An example for such an arrangement can be seen in FIGS. 4 to 6.

In the representation of FIG. 4 the area around the nose 16 of the patient 14 is shown. There, a second connector part 26 is attached under the patient's skin 32 directly on a tissue anchor 34. Accordingly, the tissue anchor 34 comprises the second connector part 26. The tissue anchor 34 itself is attached to the patients bone tissue 36 of the skull. Therefore, the tissue anchor 34 is designed as a bone anchor.

The second connector part 26 comprises in this embodiment a small iron plate 38. Iron is a ferromagnetic material and therefore magnetically responsive. It goes without mentioning that aside from this exemplary use of iron, any other ferromagnetic or ferrimagnetic material that is preferably bio-acceptable will work in the same way and has to be considered as being part of the invention as well. Also, the usage of a magnet as a second connector part 26 lies within the scope of the present invention.

The location of the second connector part 26 may be chosen as needed. However, in order to achieve an optimal transmission of the magnetic field the amount of skin and soft tissue lying between the second connector part 26 and the first connector part 24 should be as low as possible. Therefore locations as the upper and lower jaw or the skull bone are preferred.

In order to avoid the need of strong forces to attach the patient interface 12 to the patient 14, the patient interface 12 is preferably designed as light as possible.

Tissue anchor 34 and patient interface device 10 are part of a patient interface system. Accordingly, connector pairs 22, 22′ and 22″, formed by first connector parts 24 and second connector parts 26, are part of the patient interface system.

Aside from using three connector pairs 22, 22′ and 22″, any other number of connector pairs 22 together with their according first and second connector parts 24 and 26 that provides sufficient hold of the patient interface 12 on the patient 14 has to be regarded as part of the invention.

Further, embodiments wherein the elements of the first and second connector parts 24, 26 are interchanged will work and lie also within the scope of the present invention. However, the preferred embodiments comprise a first connector part 24 comprising a magnet 30 arranged at the patient interface 12 and a second connector part 26 comprising a magnetically responsive element arranged on the patient 14 via or as an tissue anchor 34 as described.

The tissue anchor 34 is designed to accommodate the second connector part 26, i.e. the iron plate 38 in this embodiment. For this, the tissue anchor 34 comprises two notches 40, 40′. The notches 40 and 40′ are arranged on a base plate 42 of the tissue anchor 34 in a way that the iron plate 38 fits under them and is held in this fitting position on the base plate 42 of the tissue anchor 34. The notches 40 and 40′ together with the base plate 42 form a docking station 43.

The fixation of the tissue anchor 34 is in this particular embodiment realized by gluing the tissue anchor 34 on the underlying bone tissue 36. This can for example be done by gluing the base plate 42 directly on the bone tissue 36. It goes without saying that other ways of fixing the tissue anchor 34 to the bone tissue 36 are also possible and lie within the scope of the present invention. Examples for such other ways of fixation are using bone screws, cements or staples. Also a design of a tissue anchor 34 that already comprises bone plugs that can be inserted in preferably pre-drilled holes is possible and part of the invention.

The tissue anchor 34 or docking station 43, as well as the fixation parts mentioned before and used for fixing the tissue anchor 34 on the bone tissue 36, are preferably designed of a non-conductive material. An example for such a non-conductive material is a polymer. With the usage of such materials it is possible for the patient to get any magnetically responsive element temporally removed. This can for example be of help if there is a longer pause in the therapy for which the patient interface device 10 is used, meaning that the patient 14 does not need to wear the patient interface 12 for a longer time. During this time, metallic or magnetic parts implanted under the skin might cause a certain inconvenience, e.g. when passing metal detectors or when working or dealing with magnetic sensitive things, like computer discs or the like. Further, even a short term removal of the magnetically responsive element is possible. This is for example necessary when an MRI of the patient 14 needs to be taken. In all these cases a surgeon can easily retrieve the magnetically responsive element, i.e. the second connector part 26 in this embodiment, by placing a small incision in the patient's skin 32 in order to access the tissue anchor 34 and remove the element. For replacement of the magnetically responsive element the surgeon basically inverses this procedure.

The first placement of the tissue anchor 34 with the second connector part 26 works in a similar way. Only an insignificantly larger incision in the skin 32 might be needed. Also, where necessary depending on the fixation method, a placement of holes, screws and/or staples has to be done. Due to the relatively small tissue anchor 34 and second connector part 26 such a procedure goes comparably fast and does not require long healing terms.

In the application of the patient interface device 10, after the placement of the second connector part 26 at the patient 14 one just needs to place the patient interface 12 on the patient's face. Due to the magnetic field between the magnets 30 and the magnetically responsive elements under the patient's skin 32, i.e. the second connector parts 26, an attractive force is the result. This force keeps the patient interface 12 without the need of additional straps on the patient's face.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. Patient interface device comprising:
a patient interface (12) for delivering a gas to a patient (14), and
at least one first connector part (24) of at least one connector pair (22), said at least one connector pair (22) comprising two connector parts (24, 26) and being provided for arranging said patient interface (12) to the patient (14),
wherein said at least one first connector part (24) comprises at least one magnet (30) or at least one magnetically responsive element (38), and
wherein said at least one first connector part is adapted to magnetically interact with at least one second connector part which is comprised by a tissue anchor which is adapted to be arranged subcutaneously or adapted to be attached to bone tissue of the patient.

2. Patient interface device of claim 1, wherein the at least one first connector part (24) comprises a plurality of magnets (30) or a plurality of magnetically responsive elements (38).

3. Patient interface device of claim 1, wherein the at least one magnetically responsive element (38) of the at least one first connector part (24) comprises a ferromagnetic or ferrimagnetic material.

4. Patient interface device of claim 1, wherein the at least one magnet (30) is at least one permanent magnet or at least one electro-magnet.

5. Patient interface device of claim 1, wherein the at least one first connector part (24) is arranged at the patient interface (12).

6. Patient interface device of claim 1 comprising a plurality of first connector parts (24) of a plurality of connector pairs (22).

7. Tissue anchor being fixable to a patient, comprising:
at least one second connector part (26) of at least one connector pair (22), said at least one connector pair (22) comprising two connector parts (24, 26) and being provided for arranging a patient interface (12) to the patient (14),
wherein said at least one second connector part (26) comprises at least one magnet (30) or at least one magnetically responsive element (38), and
wherein the tissue anchor is adapted to be arranged subcutaneously or adapted to be attached to bone tissue.

8. Tissue anchor of claim 7, wherein the at least one second connector part (26) comprises a plurality of magnets (30) or a plurality of magnetically responsive elements (38).

9. Tissue anchor of claim 7, wherein the at least one magnetically responsive element (38) of the at least one second connector part (26) comprises a ferromagnetic or ferrimagnetic material.

10. (canceled)

11. (canceled)

12. Tissue anchor of claim 7, further comprising at least one docking station (43) being adapted to be attached to bone tissue (36),
wherein the at least one second connector part (26) is releasable accommodated by the docking station (43).

13. Tissue anchor of claim 12, wherein the docking station (43) is composed of non conductive materials.

14. Patient interface system comprising:
a patient interface device of claim 1.

15. Method for attaching a patient interface device of claim 1, comprising:
arranging the patient interface (12) at the patient (14) and
magnetically connecting at least one first connector part (24) of at least one connector pair (22) to at least one second connector part (26) of said at least one connector pair (22),
wherein said at least one first connector part (24) is comprised by said patient interface device (10) and said at least one second connector part (26) is comprised by a tissue anchor (34), said tissue anchor (34) being arranged subcutaneously or attached to bone tissue of the patient.
